(19) United States
(12) Patent Application Publication
(10) Pub. No.: US 2025/0382248 A1
(43) Pub. Date: Dec. 18, 2025

(54) Molten metal recuperator to facilitate heat provision for hydrocarbon pyrolysis combined with biomass/MSW gasification

(71) Applicant: Massachusetts Institute of Technology, Cambridge, MA (US)

(72) Inventors: Daniel R. Cohn, Bridgton, ME (US); Leslie Bromberg, Sharon, MA (US); Emre Gencer, Medford, MA (US); Mohammad Ostadi, Somerville, MA (US)

(21) Appl. No.: 19/235,041

(22) Filed: Jun. 11, 2025

Related U.S. Application Data
(60) Provisional application No. 63/659,668, filed on Jun. 13, 2024.

(51) Int. Cl. C07C 29/151

(52) U.S. Cl. CPC C07C 29/1518

(57) ABSTRACT

A method and apparatus are described for use in biomass/MSW gasification to provide the heat required for the pyrolysis process. This apparatus provides heat for a pyrolysis unit through combination with the biomass gasifier, improving the efficiency of the system. Novel approaches for heating the pyrolysis unit are proposed. In some of these approaches, the hot syngas from the gasifier directly or indirectly heats the molten media in the pyrolysis reactor to enable the pyrolysis. In some systems, the molten media is circulated between two different reactors. In other systems, the inputs to two different reactors is switches periodically.

This application claims priority of U.S. Provisional Patent Application Ser. No. 63/659,668, filed Jun. 13, 2024, the disclosure of which is herein incorporated by reference in its entirety.

### Background

Low-carbon fuels are required to reduce emissions in the transportation sector as well as other difficult to electrify sectors. Biomass as a renewable carbon source plays a significant role in producing this low-carbon fuel. However, there is a challenge here: biomass is hydrogen-deficient and when used in a gasifier to produce syngas, the syngas is also hydrogen-lean. In a biomass-to-liquid (BTL) process, hydrogen-rich syngas is usually required (e. g. H₂/CO˜2 for Fischer-Tropsch and Methanol synthesis). To overcome this challenge of hydrogen-deficiency in the syngas, usually a water-gas-shift (WGS) reactor is used, which increases the H₂/CO ratio, but at a cost of carbon loss from the process. That is the main reason that BTL processes have low carbon efficiency. Another way to increase H₂ content in the syngas is to add external H₂ to the process. This external H₂ stream must preferentially be made without CO₂ emissions to the atmosphere. One promising approach to H₂ production aside from the well-known water electrolysis is methane pyrolysis (Eq.1). In this endothermic reaction, methane (or other hydrocarbons) is decomposed into solid carbon and H₂. Unlike other methods of H₂ production from fossil sources (such as Steam Methane Reforming (SMR)), in this approach, no direct CO₂ is emitted to the atmosphere. The produced carbon is in solid form and is a valuable byproduct. The byproduct carbon can be used in production of tires, paints, various electronic pieces, etc. Depending on the quality of the produced carbon, its price can reach more than 100 000 $/tonne C.

$${CH}_{4} ( g ) \to C ( s ) + 2 {H}_{2} ( g ) {Δ}_{R} H ° = 74.91 kJ {mol}^{- 1} \qquad ( Eq . 1 )$$

Although Equation 1 is written for methane, the pyrolysis reaction can be used with any hydrocarbon or hydrocarbon mixture. In particular, natural gas (which primarily comprises methane) can be used.

Due to the mildly endothermic nature of methane pyrolysis reaction (Eq.1), the provision of heat is of great importance which can affect the carbon and thermal efficiencies of the process. In the absence of a catalyst, temperatures above 700° C. are needed for the pyrolysis reaction to happen, while the presence of a catalyst reduces the reaction temperature. To have acceptable methane conversion rates, higher temperatures are required, such as 1000° C. for thermal processes and 2000° C. for plasma routes.

In thermochemical pathways for fuel production from biomass, a gasifier is used which, depending on its type, can operate between 400°° C. to 1700° C. There are advantages and disadvantages with operating at low or high temperatures. One of the main challenges at low temperatures is high tar and methane content in the produced syngas stream. On the other hand, at high temperatures the cost of the gasifier and provision of oxidant (O₂) increases.

Therefore, a system that uses a gasifier to provide the heat needed for pyrolysis would be beneficial.

### Summary

A method and apparatus are described for use in biomass/MSW gasification to provide the heat required for the pyrolysis process. This apparatus provides heat for a pyrolysis unit through combination with the biomass gasifier, improving the efficiency of the system. Novel approaches for heating the pyrolysis unit are proposed. In some of these approaches, the hot syngas from the gasifier directly or indirectly heats the molten media in the pyrolysis reactor to enable the pyrolysis. In some systems, the molten media is circulated between two different reactors. In other systems, the inputs to two different reactors is switches periodically.

In one embodiment, a process for production of methanol is disclosed. The process comprises creating syngas using a gasifier, the syngas comprising hydrogen and carbon oxides; generating a separate hydrogen stream by pyrolysis of a hydrocarbon feedstock; and converting the syngas and separate hydrogen stream to raw methanol in a methanol synthesis reaction. In some embodiments, creating syngas comprises: providing a biomass/MSW (municipal solid waste) feedstock; preparing a separate oxygen stream by separation of air; introducing the separate oxygen stream into a gasifier; and gasifying, in the gasifier, the biomass/MSW feedstock to produce the syngas. In certain embodiments, the separation of air is powered by renewable energy. In some embodiments, module (M=(H₂−CO)/(CO+CO₂)) of the syngas is adjusted to a value of between 1.9 and 2.2 by adding a part of the separate hydrogen stream into the syngas. In some embodiments, the syngas exiting the gasifier is hot syngas, and wherein the pyrolysis is heated through heat exchange with the hot syngas. In certain embodiments, a flow of hydrocarbon feedstock and hot syngas is controlled so that available heat from the hot syngas exceeds heat required to pyrolyze the hydrocarbon feedstock. In certain embodiments, additional heating for pyrolysis is provided by renewable energy or by burning part of the separate hydrogen stream or by burning part of hydrocarbon feedstock. In some embodiments, the syngas exiting the gasifier is hot syngas, and wherein the hot syngas enters a first reactor, and the pyrolysis is conducted in a second reactor, wherein a molten media flows between the first reactor and the second reactor to cool the hot syngas and supply heat for the pyrolysis. In some embodiments, the pyrolysis is conducted in a reactor containing a molten media and hot syngas exiting the gasifier indirectly heats the molten media. In some embodiments, the pyrolysis is conducted in a reactor containing a molten media, and hot syngas exiting the gasifier and the hydrocarbon feedstock are introduced into the reactor. In some embodiments, a first reactor and a second reactor both contain a molten media, and the hydrocarbon feedstock and hot syngas exiting the gasifier are switched between the first reactor and the second reactor such that the molten media is heated by the hot syngas in one reactor while pyrolysis of the hydrocarbon feedstock is being performed in the other reactor.

According to another embodiment, for creating methanol is disclosed. The system comprises a gasifier, accepting biomass/MSW (municipal solid waste) feedstock as inputs and producing hot synthetic gas, also referred to as hot syngas; a pyrolysis reactor, containing a molten media, the pyrolysis reactor accepting a hydrocarbon feedstock as an input and generating hydrogen through pyrolysis, wherein the molten media is heated directly or indirectly by the hot syngas, which, in turn, cools the hot syngas; and a methanol reactor wherein the hydrogen and cooled syngas are converted into methanol. In some embodiments, the hot syngas passes through a regenerator, which is used to heat the molten media in the pyrolysis reactor. In some embodiments, the system comprises a syngas cleaning reactor, containing molten media, wherein the hot syngas enters the syngas cleaning reactor, heats the molten media, and the molten media is exchanged between the pyrolysis reactor and the syngas cleaning reactor. In some embodiments, the hot syngas enters the pyrolysis reactor, which serves to heat the molten media, and cool the hot syngas.

According to another embodiment, a system for creating methanol is disclosed. The system comprises a gasifier, accepting biomass/MSW (municipal solid waste) feedstock as inputs and producing hot synthetic gas, also referred to as hot syngas; a first reactor, containing a molten media; a second reactor, containing a molten media; high temperature switching valve, having an input to receive the hot syngas and two outputs, a first output in communication with the first reactor and a second output in communication with the second reactor, such that when in a first position, the hot syngas enters the first reactor and when in a second position, the hot syngas enters the second reactor; a second high temperature switching valve, having an input to receive a hydrocarbon feedstock and two outputs, a first output in communication with the first reactor and a second output in communication with the second reactor, such that when in the first position, the hydrocarbon feedstock enters the second reactor and when in the second position, the hydrocarbon feedstock enters the first reactor; a controller to periodically switch the first high temperature switching valve and second high temperature switching valve between the first position and the second position; and a methanol reactor wherein hydrogen and cooled syngas produced by the first reactor and the second reactor are converted into methanol. In some embodiments, when in the first position, syngas cleaning is performed in the first reactor and pyrolysis is performed in the second reactor, and wherein the controller switches the first high temperature switching valve and second high temperature switching valve to the second position when a temperature of the molten media in the second reactor approaches a minimum temperature needed for pyrolysis.

### BRIEF DESCRIPTION OF THE DRAWINGS

For a better understanding of the present disclosure, reference is made to the accompanying drawings, in which like elements are referenced with like numerals, and in which:

FIG. 1 shows a molten media recuperator integrated in the biomass-to-liquid (BTL) process.

FIG. 2 shows a molten media recuperator integrated in the biomass-to-liquid (BTL) process with a single reactor. The hot syngas does not mix at any time with the molten media.

FIG. 3 shows a molten media recuperator integrated in the biomass-to-liquid (BTL) process using a single reactor, where both hot syngas and the hydrocarbon are introduced.

FIGS. 4A-4B show a molten media regenerator integrated in the biomass-to-liquid (BTL) process with two reactors with switching reactants (hydrocarbon and hot syngas) and without the flow of molten media from one reactor to the other. However, high temperature valves are required.

FIG. 5 shows the average temperature in two regenerator-reactors configuration of FIG. 4 with switching reactants.

### DETAILED DESCRIPTION

A new method is proposed herein to provide heat for the pyrolyzer, using the excess heat from the gasification unit. The pyrolyzer includes a liquid phase high temperature media, which may be a molten salt or a molten metal, among others. The uses biomass/MSW (municipal solid waste) gasification feedstock and an oxygen stream. The oxygen stream may be created by the separation of air, such as using an air separation unit. In some embodiments, the separation of air is powered by renewable energy. In all of the embodiments, the oxygen 1 and the biomass 2 are introduced into a gasifier 12 to generate synthetic gas, which is a mixture of carbon oxides and hydrogen.

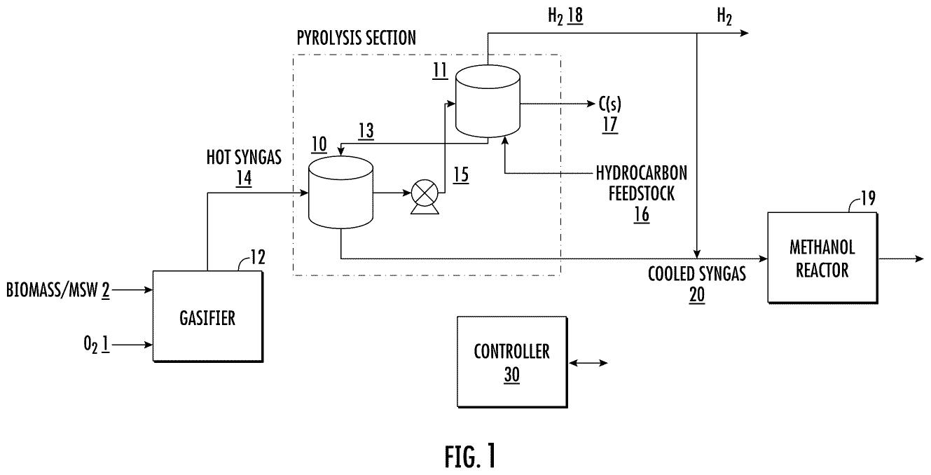

One first embodiment shown in FIG. 1 comprises two separate reactors 10, 11 where there is heat exchange between two separate gaseous streams and liquid phases. The circulating media between reactors is molten media (e.g. molten metal, molten salt, etc.). The molten media, which may be molten metals (Ti, Pb, Sn, Ga), molten metal alloys (Ni—Bi, Cu—Bi), or molten salts (KBr, NaBr, NaCl, NaF, MnCl₂, KCl), provide an effective heat transfer medium and can potentially serve as a catalyst. Typical operating temperature may be about 700-1100° C. In the syngas cleaning reactor 10, hot syngas 14 exiting the gasifier 12 is cooled through heat exchange with cool molten media 13. In this syngas cleaning reactor 10, having direct contact between phases increases the heat transfer, cooling the hot syngas 14 and heating the cool molten media 13. The cool molten media 13 can also act as a filter to convert the tars to smaller molecules and further clean the hot syngas 14, or further convert the feedstocks to hydrogen and CO (for example, converting any remaining hydrocarbon feedstock). The heated molten media 15 is then pumped to a second reactor, referred to as the pyrolysis reactor 11, where the pyrolysis takes place. The pyrolysis uses a hydrocarbon feedstock 16, such as methane, as its input. While methane is described, it is possible to use any hydrocarbon in place of methane, including natural gas, ethane, propane and other hydrocarbons, including liquid hydrocarbons, or a combination of the above.

In this pyrolysis reactor 11, hydrocarbon feedstock 16 bubbles through the heated molten media 15, cooling the heated molten media 15 because of the endothermicity of the reaction. The reaction products are carbon 17 and hydrogen 18. After separation of hydrogen, it is added to the cleaned and cooled syngas 20 that exits the syngas cleaning reactor 10 to be pressurized and sent to the methanol reactor 19. Methanol is industrially produced via catalytic conversion of syngas at pressures up to 250 bar and temperatures from ˜250° C. to 300° C. The methanol-synthesis reaction is mildly exothermic; the reactor temperature is often controlled by circulating boiling water as a coolant at elevated temperatures, like 250° C. Due to low conversion per pass in the methanol reactor, a recycle stream may be required to increase the overall conversion of the syngas. In some embodiments, the module (defined as M=(H₂−CO)/(CO+CO₂)) of the synthesis gas is adjusted to a value of between 1.9 and 2.2, by adjusting the flow rate of the hydrocarbon feedstock 16. The cool molten media 13 is then returned to the syngas cleaning reactor 10.

The molten media is removed from the syngas cleaning reactor 10 when it has reached adequate temperature, near the temperature of the hot syngas 14. In a preferred embodiment, the molten media flows in a counter flow direction with respect to the syngas, in order to provide high temperature to the molten media and to cool the syngas to temperatures that can be used in the methanol reactor 19. Additional heating or cooling of the syngas (when combined with the additional hydrogen) may be required.

For minimizing compression of the syngas, it is preferred if the gasifier 12 operates at high pressure, such as between 20 bar and 80 bar. It is, however, possible to operate at lower pressures, as low as 1 bar, with compression of the syngas after removal from the recuperator.

Similarly, the pyrolysis reactor 11 may operate at high pressure. The flows in the pyrolysis reactor 11 are also counterflow direction. The carbon 17 produced in pyrolysis reactor 11 floats to the surface and may be removed by mechanical means.

It may be necessary to balance the flow of hydrocarbon feedstock 16 and hot syngas 14 so that the available heat from the hot syngas 14 exceeds the heat required to pyrolyze the hydrocarbon feedstock 16. The flow of molten media may also to be adjusted for best operation. A controller 30 may be used to adjust the temperature and flow rate of the molten media (i.e., the enthalpy flow rate) to optimize the pyrolyzing in the pyrolysis reactor 11.

Additional external energy could be provided to assure that sufficient energy is available to pyrolyze the hydrocarbon feedstock.

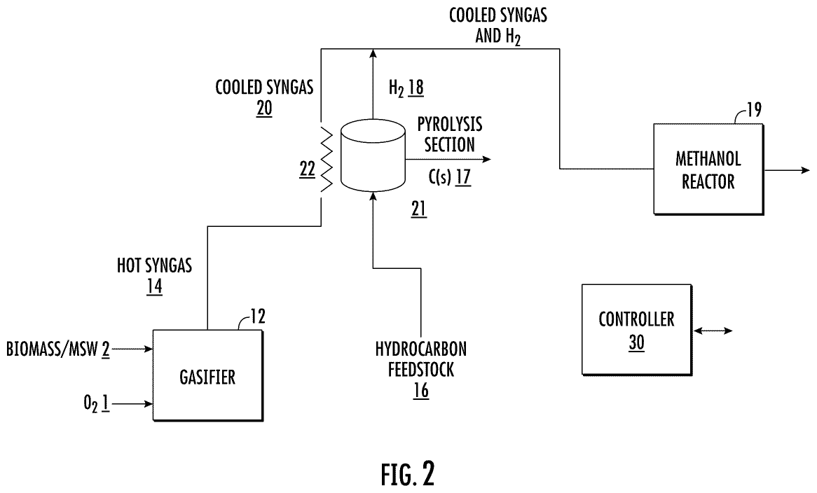

In a second embodiment shown in FIG. 2, in the pyrolysis reactor 21, there may be no direct contact between the molten media and the hot syngas 14 from the gasifier 12. The hot syngas 14 indirectly heats the molten media in the pyrolysis reactor 21 using a regenerator 22, therefore there is no concern of any transfer of contaminants from one side to the other. In case the heat from the gasifier 12 is not enough to supply the heat for the pyrolysis, the rest of the heat can be provided through other means such as renewable energy or by burning part of the hydrogen stream or by burning part of hydrocarbon feedstock 16. The rest of the system is as described above. Although the figure schematically shows the hydrocarbon feedstock 16 and the hot syngas 14 flowing in the same direction, it is not limiting. In another embodiment, there is counterflow between the hot syngas 14 and the hydrocarbon feedstock 16.

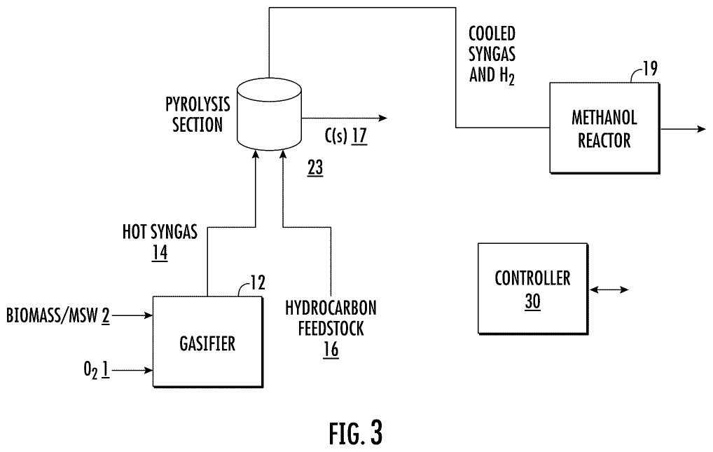

In still another embodiment shown in FIG. 3, the hot syngas 14 and the hydrocarbon feedstock 16 may be directly introduced into the molten media contained in the pyrolysis reactor 23, either mixed or unmixed with the hydrocarbon feedstock, thereby avoiding a heat exchanger. The molten media may also act as a scrubber, absorbing residual tar and carbon particles carried over from the gasifier 12. Thus, the hot syngas 14 heats the molten media while the hydrocarbon pyrolysis cools it. Both cooled syngas and hydrogen exit from the pyrolysis reactor 23 and enter the methanol reactor 19.

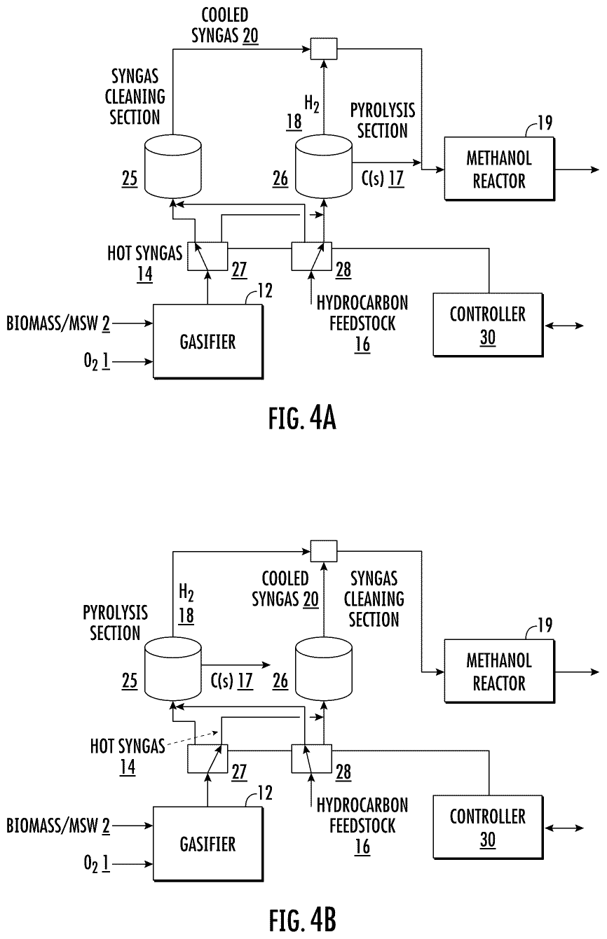

A fourth embodiment, shown in FIGS. 4A-4B, includes two reactors 25, 26. The molten media does not move from one reactor to the other reactor; rather the molten media is contained in both reactors. Instead, the hot syngas 14 and the hydrocarbon feedstock 16 switch back and forth between the two reactors via two high temperature switching valves 27, 28. In FIG. 4A, the first high temperature switching valve 27 is set to a first position so as to deliver hot syngas 14 to the first reactor 25, while the second high temperature switching valve 28 is set to a first position to deliver hydrocarbon feedstock 16 to the second reactor 26. The output from the first reactor 25 is the cooled syngas 20, while the output from the second reactor is the hydrogen 18. These are combined and enter the methanol reactor 19. In FIG. 4B, the first and second high temperature switching valves 27, 28 are set to the second position such that hot syngas 14 is delivered to the second reactor 26, while hydrocarbon feedstock 16 is delivered to the first reactor 25. Since the molten media in the first reactor 25 is still hot from the configuration shown in FIG. 4A, the hydrocarbon feedstock 16 is pyrolyzed. Meantime, the hot syngas 14 serves to heat the molten media in the second reactor 26 so that it is capable of pyrolysis when in the configuration shown in FIG. 4A. A controller 30 may be used to control the first and second high temperature switching valves 27, 28, so as to periodically switch the valves.

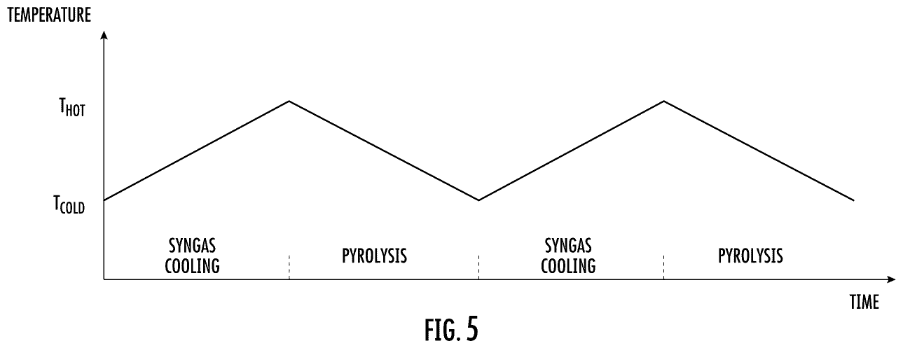

FIG. 5 shows the temperature of one of the two reactors 25, 26 in this configuration. This approach requires high temperature gas valves. Adjustment of the temperature can be achieved by setting the rate of injection of the hydrocarbon feedstock 16, hot syngas 14 or through externally cooling or heating the units. Similarly, adjustment of the hydrogen to CO ratio from the unit can be adjusted by controlling the flows of syngas, hydrocarbon feedstock or by using hydrogen or CO₂ separation units to adjust the feed ratio.

The switching frequency depends on several factors, such as the type of molten media used (e.g. Molten salt, molten metal, etc.), which dictates the temperature, gasifier scale and pyrolyzer scale. n some embodiments, the high temperature switching valves 27, 28, can switch in hourly or subhourly range, depending on the above factors. Typical operating temperature could be about 700-1100° C. The temperature excursion may be as large as 100-200° C.; however, the lower temperature should be adequate to effectively pyrolyze the hydrocarbon feedstock 16.

When the temperature is approaching that required for effectively pyrolyzing the hydrocarbon feedstock 16, the flows are switched. Effective pyrolysis may be defined when more than 80% or 90% of the hydrocarbon feedstock 16 is pyrolyzed.

The gasifier 12 may be a biomass or a MSW gasifier. However, any gaseous high temperature feedstock may be used for the heating of the gasifier, including plasma converters. The gasifier 12 and the pyrolysis units can be operated at atmospheric pressure or at elevated pressures, such as 10-50 bars.

The high temperature outputs (either the syngas, the hydrogen rich gas or both) of the pyrolysis reactor may be used to preheat the gases going to either the gasifier or to the hydrocarbons or other components of the plant, including steam generation.

Any excess heat can be used elsewhere in the process (for example, to operate a distillation column) or the generate steam to be used for driving a turbine or other processes.

While the production of methanol is described, it is understood that this system has other uses as well. For example, the process is also applicable to the production of other liquid hydrocarbons using Fischer Tropsch systems, which also require H₂ to CO ratios of 2. In this embodiment, the methanol reactor 19 is replaced with a Fischer Tropsch reactor.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A process for production of methanol, comprising:
creating syngas using a gasifier, the syngas comprising hydrogen and carbon oxides;
generating a separate hydrogen stream by pyrolysis of a hydrocarbon feedstock; and
converting the syngas and separate hydrogen stream to raw methanol in a methanol synthesis reaction.

2. The process of claim 1, wherein creating syngas comprises:
providing a biomass/MSW (municipal solid waste) feedstock;
preparing a separate oxygen stream by separation of air;
introducing the separate oxygen stream into a gasifier; and
gasifying, in the gasifier, the biomass/MSW feedstock to produce the syngas.

3. The process of claim 2, wherein the separation of air is powered by renewable energy.

4. The process of claim 1, wherein module (M=(H2−CO)/(CO+CO2)) of the syngas is adjusted to a value of between 1.9 and 2.2 by adding a part of the separate hydrogen stream into the syngas.

5. The process of claim 1, wherein the syngas exiting the gasifier is hot syngas, and wherein the pyrolysis is heated through heat exchange with the hot syngas.

6. The process of claim 5, wherein a flow of hydrocarbon feedstock and hot syngas is controlled so that available heat from the hot syngas exceeds heat required to pyrolyze the hydrocarbon feedstock.

7. The process of claim 5, wherein additional heating for pyrolysis is provided by renewable energy or by burning part of the separate hydrogen stream or by burning part of hydrocarbon feedstock.

8. The process of claim 1, wherein the syngas exiting the gasifier is hot syngas, and wherein the hot syngas enters a first reactor, and the pyrolysis is conducted in a second reactor, wherein a molten media flows between the first reactor and the second reactor to cool the hot syngas and supply heat for the pyrolysis.

9. The process of claim 1, wherein the pyrolysis is conducted in a reactor containing a molten media and hot syngas exiting the gasifier indirectly heats the molten media.

10. The process of claim 1, wherein the pyrolysis is conducted in a reactor containing a molten media, and hot syngas exiting the gasifier and the hydrocarbon feedstock are introduced into the reactor.

11. The process of claim 1, comprising a first reactor and a second reactor both containing a molten media, and wherein the hydrocarbon feedstock and hot syngas exiting the gasifier are switched between the first reactor and the second reactor such that the molten media is heated by the hot syngas in one reactor while pyrolysis of the hydrocarbon feedstock is being performed in the other reactor.

12. A system for creating methanol, comprising:
a gasifier, accepting biomass/MSW (municipal solid waste) feedstock as inputs and producing hot synthetic gas, also referred to as hot syngas;
a pyrolysis reactor, containing a molten media, the pyrolysis reactor accepting a hydrocarbon feedstock as an input and generating hydrogen through pyrolysis, wherein the molten media is heated directly or indirectly by the hot syngas, which, in turn, cools the hot syngas; and
a methanol reactor wherein the hydrogen and cooled syngas are converted into methanol.

13. The system of claim 12, wherein the hot syngas passes through a regenerator, which is used to heat the molten media in the pyrolysis reactor.

14. The system of claim 12, further comprising a syngas cleaning reactor, containing molten media, wherein the hot syngas enters the syngas cleaning reactor, heats the molten media, and the molten media is exchanged between the pyrolysis reactor and the syngas cleaning reactor.

15. The system of claim 12, wherein the hot syngas enters the pyrolysis reactor, which serves to heat the molten media, and cool the hot syngas.

16. A system for creating methanol, comprising:
a gasifier, accepting biomass/MSW (municipal solid waste) feedstock as inputs and producing hot synthetic gas, also referred to as hot syngas;
a first reactor, containing a molten media;
a second reactor, containing a molten media;
a first high temperature switching valve, having an input to receive the hot syngas and two outputs, a first output in communication with the first reactor and a second output in communication with the second reactor, such that when in a first position, the hot syngas enters the first reactor and when in a second position, the hot syngas enters the second reactor;
a second high temperature switching valve, having an input to receive a hydrocarbon feedstock and two outputs, a first output in communication with the first reactor and a second output in communication with the second reactor, such that when in the first position, the hydrocarbon feedstock enters the second reactor and when in the second position, the hydrocarbon feedstock enters the first reactor;
a controller to periodically switch the first high temperature switching valve and second high temperature switching valve between the first position and the second position; and
a methanol reactor wherein hydrogen and cooled syngas produced by the first reactor and the second reactor are converted into methanol.

17. The system of claim 16, wherein when in the first position, syngas cleaning is performed in the first reactor and pyrolysis is performed in the second reactor, and wherein the controller switches the first high temperature switching valve and second high temperature switching valve to the second position when a temperature of the molten media in the second reactor approaches a minimum temperature needed for pyrolysis.
